(19) United States
(12) Patent Application Publication
(10) Pub. No.: US 2022/0026701 A1
(43) Pub. Date: Jan. 27, 2022

(54) OPTICAL UNIT, IMAGE PICKUP APPARATUS, ENDOSCOPE, AND METHOD FOR MOVING LENS OF OPTICAL UNIT

(71) Applicant: OLYMPUS CORPORATION, Tokyo (JP)

(72) Inventor: Yuichi WATAYA, Tokyo (JP)

(73) Assignee: OLYMPUS CORPORATION, Tokyo (JP)

(21) Appl. No.: 17/493,964

(22) Filed: Oct. 5, 2021

Related U.S. Application Data
(63) Continuation of application No. PCT/JP2019/015138, filed on Apr. 5, 2019.

(51) Int. Cl. G02B 23/24; G02B 7/04; A61B 1/04; A61B 1/00

(52) U.S. Cl. CPC G02B 23/243; A61B 1/00096; A61B 1/04; G02B 7/04

(57) ABSTRACT

An optical unit includes a moving barrel made of a magnetic body and configured to hold an optical element, a fixed barrel made of a nonmagnetic body and configured to accommodate the moving barrel so as to freely move back and forth in a direction along an optical axis, a bias member configured to bias the moving barrel in a first direction along the optical axis of the lens unit, a magnet disposed on an outer circumferential surface of the fixed barrel and configured to attract the moving barrel in a second direction along the optical axis by a magnetic force, and a coil wound around the outer circumferential surface of the fixed barrel and configured to make variable the magnetic force given to the moving barrel.

### CROSS REFERENCE TO RELATED APPLICATION

This application is a continuation application of PCT/JP2019/015138 filed on Apr. 5, 2019, the entire contents of which are incorporated herein by this reference.

### BACKGROUND OF THE INVENTION

### 1. Field of the Invention

The present invention relates to an image pickup apparatus provided in an endoscope, and more specifically, to an optical unit provided at a distal end portion of an insertion portion to be inserted into a subject or object, an image pickup apparatus, an endoscope, and a method for moving a lens of the optical unit.

### 2. Description of the Related Art

As is well known, endoscopes are widely used for observation, treatment and the like of an inside of a living body (interior of body cavity) or for inspection, repair and the like of an inside of industrial plant equipment. Such endoscopes include a long insertion portion to be inserted into a body cavity, an abdominal cavity, a duct or the like of a subject or object. In recent years, electronic endoscopes with an image pickup apparatus incorporated in a distal end portion of the insertion portion have been known.

As is described in, for example, U.S. Patent Application Publication No. 2010/0127580, an image pickup apparatus provided in such an endoscope is configured to cause a movable barrel of a magnetic body to move back and forth and move a movable lens through an actuator that moves the movable barrel of the magnetic body using a magnet and an electromagnet.

As such a conventional image pickup apparatus, one provided with an actuator using a magnet and an electromagnet in a lens unit and configured to move a movable lens back and forth and realize functions such as zoom and focus is known.

### SUMMARY OF THE INVENTION

An optical unit according to an aspect of the present invention is a lens unit mounted on a distal end portion of an insertion portion of an endoscope, the lens unit including a moving barrel made of a magnetic body and configured to hold an optical element that constitutes a lens unit to form an object image, a fixed barrel made of a nonmagnetic body and configured to accommodate the moving barrel so as to freely move back and forth in a direction along an optical axis of the lens unit, a bias member configured to bias the moving barrel in a first direction along the optical axis, a magnet disposed on an outer circumferential surface of the fixed barrel and configured to attract the moving barrel in a second direction opposite to the first direction along the optical axis by a magnetic force, and a coil wound around the outer circumferential surface of the fixed barrel and configured to make variable the magnetic force given to the moving barrel by a magnetic field generated through current application.

An image pickup apparatus according to another aspect of the present invention is an image pickup apparatus mounted on a distal end portion of an insertion portion of an endoscope, the image pickup apparatus including an optical unit including a moving barrel made of a magnetic body and configured to hold an optical element that constitutes a lens unit to form an object image, a fixed barrel made of a nonmagnetic body and configured to accommodate the moving barrel so as to freely move back and forth in a direction along an optical axis of the lens unit, a bias member configured to bias the moving barrel in a first direction along the optical axis, a magnet disposed on an outer circumferential surface of the fixed barrel and configured to attract the moving barrel in a second direction opposite to the first direction along the optical axis by a magnetic force, and a coil wound around the outer circumferential surface of the fixed barrel and configured to make variable the magnetic force given to the moving barrel by a magnetic field generated through current application and an image pickup device configured to receive and photoelectrically convert light focused by the optical element.

An endoscope according to a further aspect of the present invention includes an image pickup apparatus including an optical unit, the optical unit including a moving barrel made of a magnetic body and configured to hold an optical element that constitutes a lens unit to form an object image, a fixed barrel made of a nonmagnetic body and configured to accommodate the moving barrel so as to freely move back and forth in a direction along an optical axis of the lens unit, a bias member configured to bias the moving barrel in a first direction along the optical axis, a magnet disposed on an outer circumferential surface of the fixed barrel and configured to attract the moving barrel in a second direction opposite to the first direction along the optical axis by a magnetic force, and a coil wound around the outer circumferential surface of the fixed barrel and configured to make variable the magnetic force given to the moving barrel by a magnetic field generated through current application, and an image pickup device configured to receive and photoelectrically convert light focused by the optical element, and an insertion portion with a distal end portion on which the image pickup apparatus is mounted.

A method for moving a lens of an optical unit according to a further aspect of the present invention is a method for moving a lens of an optical unit, the optical unit being mounted on a distal end portion of an insertion portion of an endoscope and including a moving barrel made of a magnetic body and configured to hold an optical element that constitutes a lens unit to form an object image, a fixed barrel made of a nonmagnetic body and configured to accommodate the moving barrel so as to freely move back and forth in a direction along an optical axis of the lens unit, a bias member configured to bias the moving barrel in a first direction along the optical axis, a magnet disposed on an outer circumferential surface of the fixed barrel and configured to attract the moving barrel in a second direction opposite to the first direction along the optical axis by a magnetic force, and a coil wound around the outer circumferential surface of the fixed barrel and configured to make variable the magnetic force given to the moving barrel by a magnetic field generated through current application, the method including moving the moving barrel in the first direction by the bias member and moving the moving barrel in the second direction by current application to the coil.

### BRIEF DESCRIPTION OF THE DRAWINGS

FIG. 1 is a diagram illustrating an appearance of an endoscope according to one aspect of the present invention;

FIG. 2 is a schematic view illustrating a configuration of a distal end portion of an insertion portion of the endoscope according to the one aspect of the present invention:

FIG. 3 is a cross-sectional view illustrating a configuration of a lens unit of the endoscope according to the one aspect of the present invention in which a movable lens barrel has moved to a distal end side:

FIG. 4 is a cross-sectional view illustrating a configuration of the lens unit of the endoscope according to the one aspect of the present invention in which the movable lens barrel has moved to a proximal end side;

FIG. 5 is a cross-sectional view illustrating a configuration of a lens unit according to a first modification in which the movable lens barrel has moved to the distal end side:

FIG. 6 is a cross-sectional view illustrating a configuration of the lens unit according to the first modification in which the movable lens barrel has moved to the proximal end side;

FIG. 7 is a cross-sectional view illustrating a configuration of a lens unit according to a second modification in which the movable lens barrel has moved to the distal end side; and

FIG. 8 is a cross-sectional view illustrating a configuration of the lens unit according to the second modification in which the movable lens barrel has moved to the proximal end side.

### DETAILED DESCRIPTION OF THE PREFERRED EMBODIMENTS

Hereinafter, an image pickup apparatus provided in an endoscope according to one aspect of the present invention will be described based on the accompanying drawings. Note that in the following description, drawings based on each embodiment are schematic ones and a thickness-width relationship among components and thickness ratios among the components are different from the actual relationships and ratios, and there are cases where dimensional relationships and ratios differ among drawings.

The endoscope given as an example in the following configuration description is a so-called flexible endoscope having an insertion portion with flexibility so as to be inserted into a bronchus, a urinary organ, or from an esophagus into a body cavity such as a stomach, a small intestine or a large intestine of a living body. However, the present invention is also applicable to a so-called rigid endoscope having a rigid insertion portion including a bending portion to be used for surgery.

An endoscope according to one aspect of the present invention will be described based on the accompanying drawings.

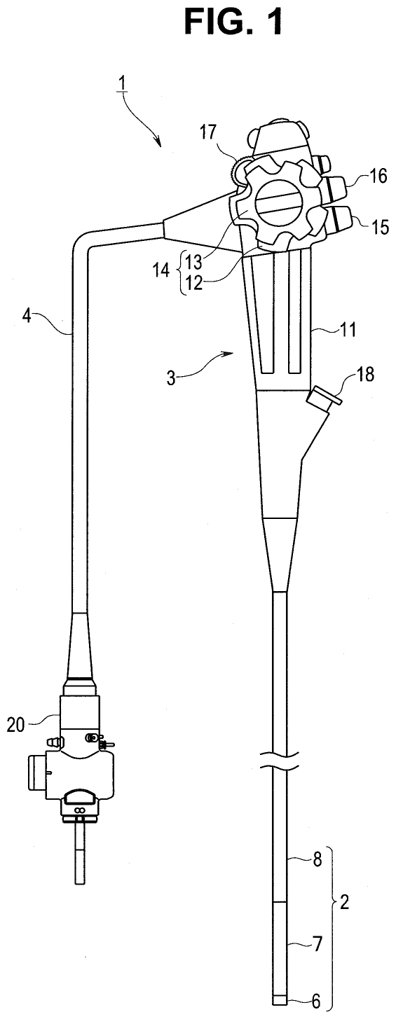

As illustrated in FIG. 1, an endoscope 1 of the present embodiment is constituted of a long elongated insertion portion 2 to be inserted into a subject or object, an operation portion 3, and a universal cable 4, which is a composite cable. The insertion portion 2 of the endoscope 1 is constituted of a distal end portion 6, a bending portion 7 and a flexible tube portion 8 in order from the distal end.

The operation portion 3 is provided with a bending operation knob 14 disposed turnably for bending the bending portion 7 of the insertion portion 2, switches 15 and 16 for switching various endoscope functions, near point observation, far point observation, release, an observation image such as a still image, and a fixing lever 17 for fixing rotation of the bending operation knob 14 or the like.

Note that the bending operation knob 14 is disposed so that two substantially disk-like rotation knobs; an UD bending operation knob 12 for bending the bending portion 7 in an up-down direction and an RL bending operation knob 13 for bending the bending portion 7 in a left-right direction overlap.

A connection portion between the insertion portion 2 and the operation portion 3 is constituted of a grasping portion 11 grasped by a user and a treatment instrument insertion channel insertion portion 18 disposed at the grasping portion 11 and configured to serve as an opening portion of a treatment instrument insertion channel that allows insertion of various treatment instruments disposed in the insertion portion 2.

The universal cable 4 that extends from the operation portion 3 includes an endoscope connector 20 which is freely attachable/detachable to/from a light source apparatus (not shown) at an extending end. Note that the endoscope 1 of the present embodiment is configured to transmit illumination light from a light source apparatus (not shown) to the distal end portion 6 through a light guide bundle (not shown), which is illumination means inserted and disposed in the insertion portion 2, the operation portion 3 and the universal cable 4.

A coil-shaped coil cable (not shown) is connected to the endoscope connector 20 and an electric connector which is freely attachable/detachable to/from a video processor (not shown) is provided at an extending end of the coil cable.

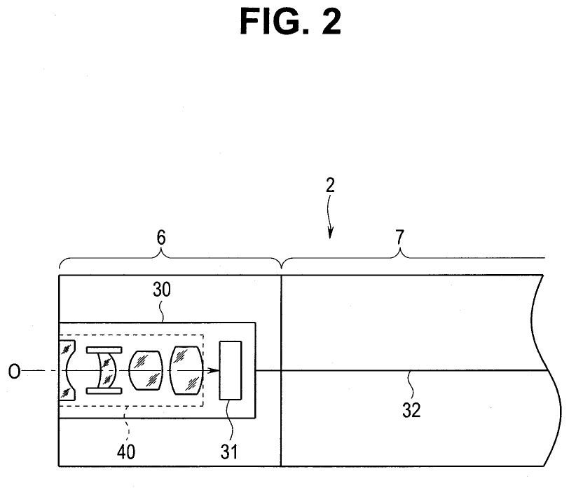

An image pickup apparatus 30 is mounted on the distal end portion 6 of the insertion portion 2 as illustrated in FIG. 2.

The image pickup apparatus 30 includes a lens unit 40, which is an optical unit and is provided with a solid image pickup device 31 such as a CCD or a CMOS configured to receive and photoelectric convert an object image formed by the lens unit 40. The lens unit 40 is constituted of at least one optical element.

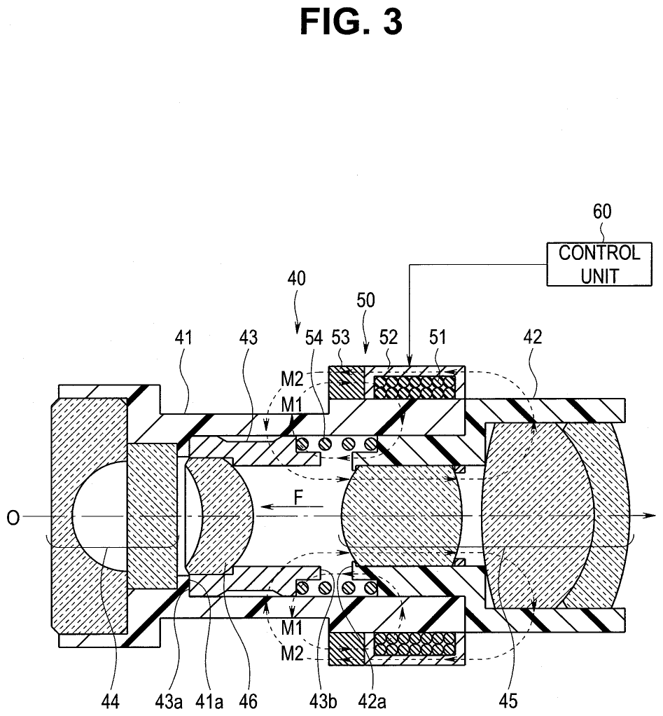

As illustrated in FIG. 3, the lens unit 40 of the present embodiment includes a first lens holding barrel 41, which is a fixed barrel on a distal end side, a second lens holding barrel 42, which is a fixed barrel engaging with a proximal end side of the first lens holding barrel 41 and a movable lens barrel 43, which is a moving barrel, accommodated so as to freely move back and forth within the first lens holding barrel 41. The movable lens barrel 43 moves along an optical axis of the lens unit 40.

The first lens holding barrel 41 is made of a nonmagnetic member such as rigid resin or nonmagnetic stainless steel. Here, the first lens holding barrel 41 holds a front group lens 44, which is a fixed lens group constituted of optical elements, which are two objective optical systems.

The second lens holding barrel 42 is also made of a nonmagnetic member such as rigid resin or nonmagnetic stainless steel. Here, the second lens holding barrel 42 holds a rear group lens 45, which is a fixed lens group constituted of optical elements, which are three objective optical systems. A circumferential groove is formed on an outer circumference of the distal end of the second lens holding barrel 42.

The movable lens barrel 43 is made of a magnetic member such as iron or nickel, and holds a movable lens 46 of an optical element, which is one objective optical system. A circumferential groove is formed on an outer circumference of the proximal end of the movable lens barrel 43.

A coil spring 54, which is a bias member, is mounted in respective circumferential grooves of the second lens holding barrel 42 and the movable lens barrel 43, and the movable lens barrel 43 is biased forward on the distal end side by the coil spring 54.

The first lens holding barrel 41 is provided with an actuator 50 on a proximal end outer circumference.

The actuator 50 includes a coil part 51 with a metal elemental wire of copper or the like wound around the proximal end outer circumference of the first lens holding barrel 41 in a predetermined direction, a metal yoke 52 of iron, copper, or the like disposed so as to cover the coil part 51 and magnets 53, which are permanent magnets, arranged side by side on the distal end side of the yoke 52.

The actuator 50 is electrically connected to a control unit 60 configured to control current application to the coil part 51. Note that the control unit 60 is provided in an external device and configured to turn ON/OFF current application to the actuator 50 according to a user operation on the switches 15 and 16 provided in the operation portion 3 of the endoscope 1.

When current is applied to the coil part 51 of the actuator 50 in the lens unit 40 of the image pickup apparatus 30 configured as described above as illustrated in FIG. 3, a magnetic field M2 in a direction opposite to a magnetic field M1 of the magnet 53 is generated in the coil part 51 and cancels the magnetic force of the magnet 53.

In this condition, the movable lens barrel 43 receives a biasing force of the coil spring 54 and is given a force F to move toward the distal end side.

A distal end face 43a of the movable lens barrel 43 comes into contact with an end face 41a, which becomes a distal end side stopper of an inward flange provided on a middle inner circumference of the first lens holding barrel 41 whereby a position of stopping movement to the distal end side is defined.

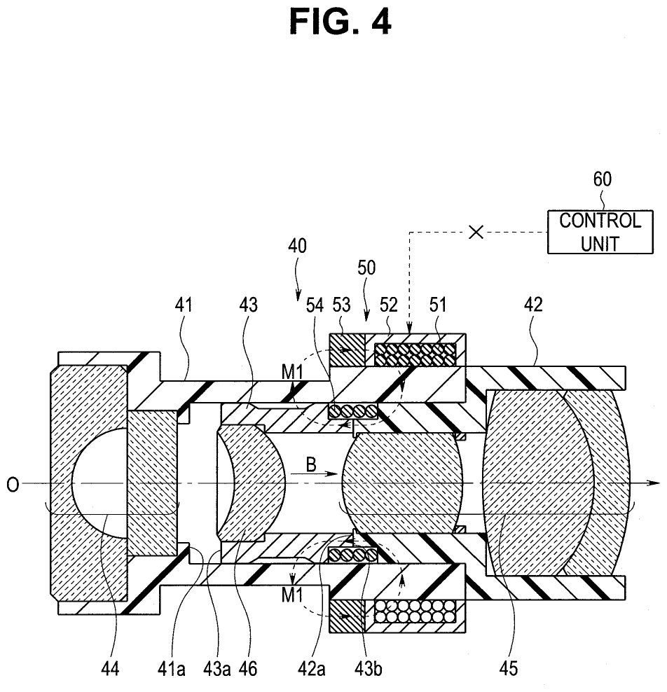

On the other hand, as illustrated in FIG. 4, when current application to the coil part 51 of the actuator 50 is stopped, there is a state in which only the magnetic field M1 of the magnet 53 is generated in the lens unit 40 of the image pickup apparatus 30.

In this condition, the movable lens barrel 43 is attracted by the magnetic force of the magnet 53 against the biasing force of the coil spring 54 toward the distal end side and is given a force B to move to the proximal end side.

A proximal end face 43b of the movable lens barrel 43 comes into contact with a distal end face 42a, which becomes a proximal end side stopper of the second lens holding barrel 42, whereby a position of stopping movement to the proximal end side is defined.

Thus, the lens unit 40 of the image pickup apparatus 30 causes the actuator 50 to generate the magnetic field M2 in the direction opposite to the magnetic field M1 that cancels the magnetic force of the magnet 53 and causes the movable lens barrel 43 made of a magnetic member that moves back and forth along an optical axis O to move to the distal end side by the biasing force of the coil spring 54.

When current application to the actuator 50 is stopped, the lens unit 40 of the image pickup apparatus 30 causes the movable lens barrel 43 made of a magnetic member to move to the proximal end side along the optical axis O against the biasing force of the coil spring 54 by the magnetic force of the magnet 53.

Note that strength of the biasing force of the coil spring 54, strength of the magnetic force of the magnet 53 and a relative position with respect to the magnet 53 are set so that the movable lens barrel 43 can move to the proximal end side by the magnetic force of the magnet 53 against the biasing force of the coil spring 54. The biasing force of the coil spring 54, the magnetic force of the magnet 53 and the strength of the magnetic field generated in the coil part 51 are respectively set so that the movable lens barrel 43 can be driven to move back and forth.

As described above, the image pickup apparatus 30 mounted on the distal end portion 6 of the insertion portion 2 of the endoscope 1 of the present embodiment is configured to control the magnetic force of the magnet 53 using a magnet 53 of the actuator 50 disposed in the lens unit 40 and cause the biasing force of the coil spring 54 to drive the movable lens barrel 43 back and forth.

This makes it possible to shorten a length of the actuator 50 in a direction along the optical axis O, shorten the lens unit 40 and reduce the size of the image pickup apparatus 30.

The actuator 50 provided in the proximal end portion of the first lens holding barrel 41 can further secure an engagement length of the distal end portion of the first lens holding barrel 41 with respect to the distal end portion 6.

Moreover, even if a large impact load is sometimes applied to the distal end portion 6 of the insertion portion 2 due to the user's handling, since the magnet 53 of the actuator 50 is incorporated in a middle position closer to the proximal end side than the distal end side of the distal end portion 6, cracks are unlikely to occur, and failure of the image pickup apparatus 30, the actuator 50 in particular, can be reduced.

As described so far, it is possible to provide the lens unit 40, the image pickup apparatus 30 and the endoscope 1 that can achieve downsizing by shortening the distal end portion 6 of the insertion portion 2 and improve resistance to an impact load to the distal end portion 6.

It is preferable to make an optical setting for the image pickup apparatus 30 so that a state in which the movable lens barrel 43 of the lens unit 40 is located on the proximal end side is set as far point observation (WIDE) and a state in which the movable lens barrel 43 is located on the distal end side is set as near point observation (TELE).

Thus, during observation using the endoscope 1, since far point observation is used more frequently, a state in which current is not applied to the actuator 50 is considered as main, and it is thereby possible to reduce power consumption and heat generation of the actuator 50 due to high temperature of the coil part 51.

Note that an optical setting may also be made for the image pickup apparatus 30 through an optical lens setting of the lens unit 40 such that a state in which the movable lens barrel 43 is located on the proximal end side is set as near point observation and a state in which the movable lens barrel 43 is located on the distal end side is set as far point observation. In addition to optical zooming whereby far point observation and near point observation are switched by causing the movable lens barrel 43 of the lens unit 40 to move back and forth, the image pickup apparatus 30 may also be configured to switch between far point observation and near point observation by focus adjustment.

Furthermore, although a case of the configuration of the lens unit 40 has been described above where the magnetic force of the magnet 53 is canceled by the magnetic field of the coil part 51 and the movable lens barrel 43 is driven to move forth by the biasing force of the coil spring 54, the magnetic force may be increased by the magnetic field of the coil part 51 in addition to the magnetic force of the magnet 53 to drive the movable lens barrel 43 to move back. With such a configuration, the biasing force of the coil spring 54 is set greater than an attractive force given to the movable lens barrel 43 by the magnetic force of the magnet 53.

### First Modification

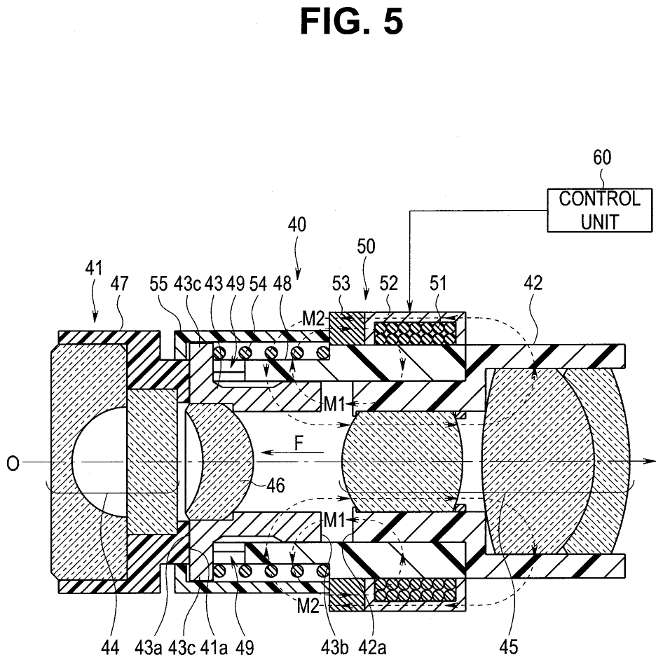

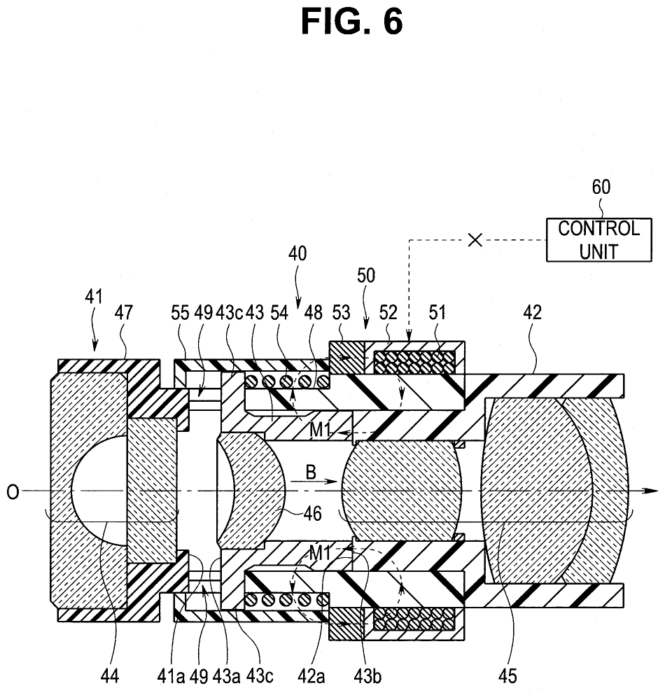

As illustrated in FIG. 5 and FIG. 6, the coil spring 54 provided in the lens unit 40 of the image pickup apparatus 30 may be configured to be fitted over an outer circumference of the first lens holding barrel 41.

The lens unit 40 here is constituted of two barrel bodies 47 and 48 with which the first lens holding barrel 41 engages, and a plurality of slits 49 are formed in a plurality of directions, for example, three directions such as up and down directions and circumferential direction or up, down, left and right directions.

In the movable lens barrel 43, a plurality of operation rods 43c, which are protruding portions that protrude from the plurality of slits 49 formed in the two barrel bodies 47 and 48 that constitute the first lens holding barrel 41 in an outer diameter direction are integrally formed.

The coil spring 54 is provided so as to come into contact with proximal end faces of the plurality of operation rods 43c and a distal end face of a swollen part of the barrel body 48 of the first lens holding barrel 41 on the proximal end side. In this way, the coil spring 54 biases the movable lens barrel 43 toward the distal end side.

The lens unit 40 is provided with a substantially cylindrical cover body 55 so as to cover the plurality of slits 49, the plurality of operation rods 43c and the coil spring 54. Note that although the cover body 55 need not always be provided, the cover body 55 provides an advantage of not allowing dust, waste or the like to enter from the plurality of slits 49 into the lens unit 40.

Note that other components and operations of the lens unit 40 are similar to the components and operations of the above embodiment.

Since the lens unit 40 in the present modification configured as described above is equipped with the coil spring 54 on the outer circumference side of the first lens holding barrel 41, in addition to the operations and effects described in the above embodiment, the lens unit 40 is easy to assemble.

Since the coil spring 54 is not housed in the lens unit 40, there are more choices for spring specifications and sliding debris generated when the coil spring 54 expands or contracts is not generated in the lens unit 40 and it is thereby possible to prevent adverse influence on images and sliding failure or the like of the movable lens barrel 43.

### Second Modification

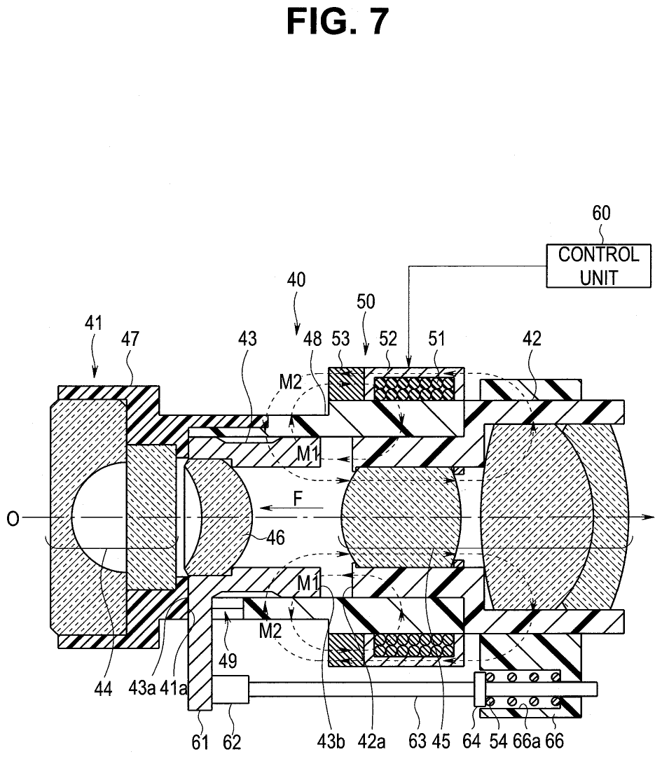

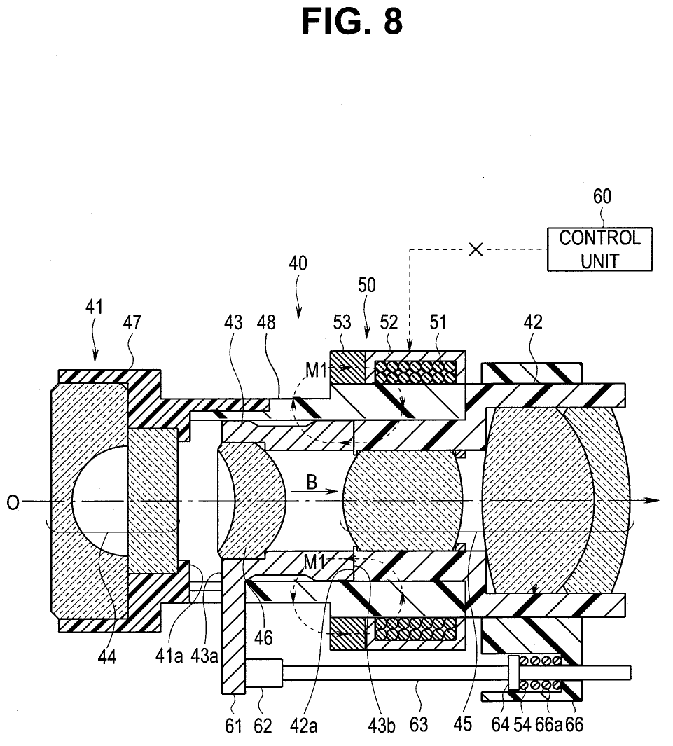

As illustrated in FIG. 7 and FIG. 8, a push rod may be adopted for the configuration in which the movable lens barrel 43 provided in the lens unit 40 of the image pickup apparatus 30 is biased to the distal end side.

Similar to the first modification, the lens unit 40 here is constituted of the two barrel bodies 47 and 48 with which the first lens holding barrel 41 engages, and one slit 49 is formed.

The movable lens barrel 43 is provided with an operation rod 61, which is integrally formed and is a protruding part that protrudes in an outer diameter direction from the one slit 49 formed in the first lens holding barrel 41.

A push rod 63 provided with a contact member 62 contacting a proximal end face of the operation rod 61 at the distal end is supported in such a way as to freely move back and forth.

A spring reception part 66 configured to accommodate the coil spring 54 to be fitted over the proximal end side of the push rod 63 and provided with a stepped hole part 66a into which a proximal end side of the push rod 63 is inserted and supported is fixed to the second lens holding barrel 42. Note that the spring reception part 66 may be integrally formed with the second lens holding barrel 42.

Note that other components and operations of the lens unit 40 are the same as the components and operations of the above embodiment.

The lens unit 40 of the present modification configured as described above is provided with the operations and effects described in the above embodiment and has a configuration in which the operation rod 61 of the movable lens barrel 43, the push rod 63, the support body 65 and the spring reception part 66 protrude only in one circumferential direction, and so the protruding parts need only to be disposed in accordance with a dead space of the distal end portion 6 and it is thereby possible to prevent the distal end portion 6 from becoming large.

The inventions described in the above embodiment and modifications are not limited to the above embodiment and modifications, but various modifications can be implemented without departing from the gist and scope of the invention in the implementation phase. Furthermore, the above embodiment and modifications include inventions in various phases and various inventions can be extracted according to appropriate combinations in a plurality of disclosed configuration requirements.

For instance, even when some configuration requirements are deleted from all the configuration requirements illustrated in the embodiment and modifications, configurations from which these configuration requirements are deleted can be extracted as inventions as long as the described problems can be solved and the described effects can be achieved.

What is claimed is:

1. An optical unit mounted on a distal end portion of an insertion portion of an endoscope, the optical unit comprising:
a moving barrel made of a magnetic body and configured to hold an optical element that constitutes a lens unit to form an object image;
a fixed barrel made of a nonmagnetic body and configured to accommodate the moving barrel so as to freely move back and forth in a direction along an optical axis of the lens unit;
a bias member configured to bias the moving barrel in a first direction along the optical axis;
a magnet disposed on an outer circumferential surface of the fixed barrel and configured to attract the moving barrel in a second direction opposite to the first direction along the optical axis by a magnetic force; and
a coil wound around the outer circumferential surface of the fixed barrel and configured to make variable the magnetic force given to the moving barrel by a magnetic field generated through current application.

2. The optical unit according to claim 1, wherein
the coil generates the magnetic field in a direction opposite to the magnetic field of the magnet and cancels the magnetic force.

3. The optical unit according to claim 1, further comprising a yoke configured to increase the magnetic field of the coil.

4. The optical unit according to claim 1, wherein
the fixed barrel comprises:
a first stopper configured to receive a biasing force of the bias member while a current is applied to the coil and come into contact with the moving barrel that moves in the first direction to define a moving position; and
a second stopper configured to receive a magnetic force of the magnet while no current is applied to the coil and come into contact with the moving barrel that moves in the second direction to define the moving position.

5. The optical unit according to claim 4, wherein the optical element is set such that a state in which the moving position of the moving barrel is defined by the second stopper becomes far point observation and a state in which the moving position of the moving barrel is defined by the first stopper becomes near point observation.

6. The optical unit according to claim 5, wherein
the bias member is disposed within the fixed barrel.

7. The optical unit according to claim 5, wherein
the bias member is fitted over an outer circumference of the fixed barrel.

8. The optical unit according to claim 1, wherein
the moving barrel comprises an operation rod that protrudes to an outside of the fixed barrel, and
the optical unit comprises a push rod configured to transmit the biasing force of the bias member, come into contact with the operation rod and press the moving barrel in the first direction.

9. An image pickup apparatus mounted on a distal end portion of an insertion portion of an endoscope, the image pickup apparatus comprising:
an optical unit comprising:
a moving barrel made of a magnetic body and configured to hold an optical element that constitutes a lens unit to form an object image;
a fixed barrel made of a nonmagnetic body and configured to accommodate the moving barrel so as to freely move back and forth in a direction along an optical axis of the lens unit;
a bias member configured to bias the moving barrel in a first direction along the optical axis;
a magnet disposed on an outer circumferential surface of the fixed barrel and configured to attract the moving barrel in a second direction opposite to the first direction along the optical axis by a magnetic force; and
a coil wound around the outer circumferential surface of the fixed barrel and configured to make variable the magnetic force given to the moving barrel by a magnetic field generated through current application; and
an image pickup device configured to receive and photoelectrically convert light focused by the optical element.

10. An endoscope comprising:
an insertion portion; and
an image pickup apparatus mounted on a distal end portion of the insertion portion, the image pickup apparatus including an optical unit,
the optical unit including:
a moving barrel made of a magnetic body and configured to hold an optical element that constitutes a lens unit to form an object image,
a fixed barrel made of a nonmagnetic body and configured to accommodate the moving barrel so as to freely move back and forth in a direction along an optical axis of the lens unit,
a bias member configured to bias the moving barrel in a first direction along the optical axis,
a magnet disposed on an outer circumferential surface of the fixed barrel and configured to attract the moving barrel in a second direction opposite to the first direction along the optical axis by a magnetic force, and
a coil wound around the outer circumferential surface of the fixed barrel and configured to make variable the magnetic force given to the moving barrel by a magnetic field generated through current application; and
an image pickup device configured to receive and photoelectrically convert light focused by the optical element.

11. A method for moving a lens of an optical unit mounted on a distal end portion of an insertion portion of an endoscope, the optical unit including
a moving barrel made of a magnetic body and configured to hold an optical element that constitutes a lens unit to form an object image;
a fixed barrel made of a nonmagnetic body and configured to accommodate the moving barrel so as to freely move back and forth in a direction along an optical axis of the lens unit;
a bias member configured to bias the moving barrel in a first direction along the optical axis;
a magnet disposed on an outer circumferential surface of the fixed barrel and configured to attract the moving barrel in a second direction opposite to the first direction along the optical axis by a magnetic force; and
a coil wound around the outer circumferential surface of the fixed barrel and configured to make variable the magnetic force given to the moving barrel by a magnetic field generated through current application,
the method comprising:
moving the moving barrel in the first direction by the bias member and moving the moving barrel in the second direction by current application to the coil.
